(19) United States
(12) Patent Application Publication
(10) Pub. No.: US 2007/0141663 A1
(43) Pub. Date: Jun. 21, 2007

(54) Process for the preparation of copolymer-1

(76) Inventors: Jinguo Ding, Shanghai (CN); Laigen Xu, Shanghai (CN); Haoyue Wang, Shi Jia Zhuang City (CN); Mingfang Ji, Kun Shan City (CN); Jiahao Shi, Shanghai (CN); Cai'e Ju, Shanghai (CN); Wai Hong Chan, San Mateo, CA (US)

Correspondence Address: COHEN, PONTANI, LIEBERMAN & PAVANE, 551 FIFTH AVENUE, SUITE 1210, NEW YORK, NY, 10176, US

(21) Appl. No.: 11/504,793

(22) Filed: Aug. 15, 2006

Related U.S. Application Data
(60) Provisional application No. 60/708,218, filed on Aug. 15, 2005.

(51) Int. Cl. C12P 21/06; C07K 14/00

(52) U.S. Cl. 435/68.1; 530/350

(57) ABSTRACT

Copolymer-1 is a mixture of synthetic polypeptides composed of alanine, glutamic acid, lysine, and tyrosine. The invention relates to an improved process for the preparation of copolymer-1 characterized by the deblocking of the protected copolymer-1 that is carried out in one reaction. The process of the present invention has the advantage of high yield and ease of production. Copolymer-1 is a useful drug in treating multiple sclerosis.

### RELATED APPLICATIONS

This application claims priority from U.S. Provisional Patent Application Ser. No. 60/708,218 which was filed on Aug. 15, 2005. The content of this provisional patent application is hereby incorporated by reference.

### BACKGROUND OF THE INVENTION

1. Field of the Invention

The present invention relates to an improved process for the preparation of copolymer-1. The structural formula is: Poly [L-Ala^{w}, L-Glu^{x}, L-Lys^{Y}, L-Tyr^{z}]·n (CH₃CO₂H), wherein w, x, y, z is between 0 with 1. Preferably, the copolymer-1 has a molar ratio of L-Ala:L-Glu:L-Lys:L-Tyr approximately 0.427:0.150:0.327:0.100, and the deviation may vary by about ±10%.

2. Description of the Related Art

Copolymer-1 is used in immunotherapy for multiple sclerosis. It is a mixture of synthetic polypeptides composed of alanine, glutamic acid, lysine, and tyrosine. A process is known for preparing copolymer-1 (U.S. Pat. No. 3,849,550), in which the N-carboxyanhydrides of tyrosine, alanine, γ-benzyl glutamate and ε-N-trifluoro-acetyl lysine are polymerized in anhydrous dioxane with diethylamine as initiator. The deblocking of the γ-carboxyl group of the glutamic acid is effected by hydrogen bromide in glacial acetic acid and is followed by the removal of the trifluoroacetyl groups from the lysine residues by 1 M piperidine.

According to the known process, the removal of the benzyl ester and N-trifluoroacetyl protection groups require two separate deblocking. The copolymer-1 was isolated by a tedious dialysis method to remove piperidine and its trifluoroacetyl derivative and to convert the copolymer-1 to the acetate salt. Furthermore, the unprotected phenol group in the tyrosine N-carboxyanhydride side chain complicates the polymerization by reacting with amine initiator to form a nucleophilic phenolate anion.

Therefore there is a need to improve the existing process for a more economic and simpler commercial synthesis.

### SUMMARY OF THE INVENTION

The process of this application describes a method for the preparation of copolymer-1 and specifically copolymer-1 with the desired amino acid composition and molecular weight distribution. By virtue of this novel method, the two separate steps for obtaining non-protected copolymer-1 in the prior art were reduced to one simple step. The method consists of copolymerization of N-Carboxyanhydride (NCA) of alanine (Ala-NCA), γ-benzyl glutamate [Glu(OBzl)-NCA], ε-N-Benzyloxycarbonyl lysine [Lys(Z)-NCA] and O-benzyl tyrosine [Tyr(Bzl)-NCA] in an inert solvent with a initiator. The choice of Tyr(Bzl)-NCA provides the advantage of being stable, crystalline and easy to obtain in high purity. The copolymerization involving the four amino acid NCAs and diethylamine offers copolymer-1 with reproducible amino acids composition and molecular weight distribution. After the completion of the polymerization, water was added to the reaction mixture to precipitate the fully protected copolymer-1. All the protecting groups on the corresponding protected copolymer-1 can be removed by hydrogen bromide in glacial acetic acid in only one step. Upon the completion of the de-protection, excess hydrobromic acid and acetic acid was removed to give a crude copolymer-1 as hydrobromic acid salt. The crude copolymer-1 HBr salt was dissolved in 1N acetic acid and purified by Sephadex G25 to remove the small molecular weight material. The purified copolymer-1 HBr salt was treated with sodium carbonate to pH 8-9 then acidify to pH 3-4 by acetic acid to convert the HBr salt to copolymer-1 acetic acid salt. Copolymer-1 acetic acid salt can be further purified by Sephadex G50 eluting with 1N acetic acid to collect the copolymer-1 acetic salt with the desired molecular weight range. Good yields of copolymer-1 acetic acid salt can be obtained in such a manner.

It also has been found that the removal of the protecting group, γ-benzyl group on glutamic acid or O-benzyl group on tyrosine, needs longer period to be removed by hydrogen bromide in glacial acetic acid. Another method is developed to obtain copolymer-1 from its protected precursor under moderate condition with higher efficiency and by using one step. The method consists of copolymerization of N-Carboxyanhydride of alanine (Ala-NCA), γ-t-butyl glutamate [Glu(OBut)-NCA], ε-N-t-butyloxycarbonyl lysine [Lys(Boc)-NCA] and O-t-butyl tyrosine [Tyr(But)-NCA] in an inert solvent with a initiator. The copolymerization involving the four amino acid NCAs and diethylamine offers copolymer-1 with reproducible amino acids composition and molecular weight distribution. After the completion of the polymerization, water was added to the reaction mixture to precipitate the fully protected copolymer-1. All the protecting groups on the corresponding protected copolymer-1 can be removed by hydrogen chloride in glacial acetic acid in only one step. Upon the completion of the de-protection, excess hydrobromic acid and acetic acid were removed to give a crude copolymer-1 as hydrochloric acid salt. The crude copolymer-1 HCI salt was dissolved in 1N acetic acid and purified by Sephadex G25 to remove the small molecular weight material. The purified copolymer-1 HCI salt was treated with sodium carbonate to pH 8-9 then acidified to pH 3-4 by acetic acid to convert the HBr salt to copolymer-1 acetic acid salt. Copolymer-1 acetic acid salt can be further purified by Sephadex G50 eluting with 1N acetic acid to collect the copolymer-1 acetic salt with the desired molecular weight range. Good yields of copolymer-1 acetic acid salt can be obtained in such a manner. The hydrogen chloride in glacial acetic acid can be replaced with trifluoroacetic acid, hydrogen chloride in dioxane or ethyl acetate

All the amino acid NCAs can be prepared by reaction of the corresponding N-butyloxycarbonyl-amino acid with triphosgene and triethylamine in a solvent medium [J. Org. Chem. 1992, 57, 2755-2756]. Ala-NCA, Glu(OBzl)-NCA, Lys(Z)-NCA and Tyr(Bzl)-NCA can be also prepared by reaction of the corresponding N-unprotected amino acid with phosgene, diphosgene or triphosgene [Tetrahedron Letters 1988, 29, 5859-5862].

In point of fact, the reaction conditions of amino acid NCAs synthesis are similar. In order to reduce the production cost of copolymer-1, it is possible to use a mixture of alanine, γ-benzyl glutamate, ε-N-Benzyloxycarbonyl lysine and 0-benzyl tyrosine as starting compounds instead of the amino acid NCAs. In one reactor, the amino acids mixture can be converted to the corresponding amino acid NCAs mixture by the same reaction. The amino acid NCAs can be converted to copolymer-1 in the subsequent copolymerization. In the same way, the mixture of alanine, ε-t-butyl glutamate, ε-Nt-butyloxycarbonyl lysine and O-t-butyl tyrosine can also be used as starting compounds directly.

The polymerization of NCAs can be carried out by simply mixing the above four NCAs in a solvent such as dioxane, tetrahydrofuran, dichloromethane, dimethylformamide, N-methylpyrrolidone, sulfolane, nitrobenzene, tetramethylurea, dimethylsulfone or other inert solvents that are capable of dissolving NCAs and results in a homogeneous reaction.

The reaction was initiated by addition of an initiator solution. Organic amine is a preferred initiator. The molar ratio of initiator to total NCA used is in the range of 0.7% to 5%. The reaction can be carried out at any convenient temperature but temperatures between 0-50° C. are preferred. Other initiators include sodium methoxide, sodium t-butoxide, hexylamine, phenethylamine or transition metal initiator such as bbyNi(COD), (Pme3)4Co.

### BRIEF DESCRIPTION OF THE FIGURES

FIG. 1 shows the elution profile of copolymer-1 HBr that has passed through a Sephadex G25 column.

FIG. 2 shows the elution profile of copolymer-1 acetate that has passed through a Sephadex G-50 column.

The following non-limitive examples illustrate the invention.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein. The following non-limiting examples illustrate the invention.

### EXAMPLE 1

General Procedure for N-Carboxy Anhydride Preparation and Purification

Amino acids and triphosgene was suspended in dry ethyl acetate or tetrahydrofuran at room temperature. The resulting mixture was stirred at 50-60° C. until a homogeneous solution was obtained. N-Hexane was added to the reaction mixture to precipitate the desired N-carboxy anhydride. The crude N-carboxy anhydride was dissolved in ethyl acetate and any undissolved material was removed by filtration. N-Hexane was added to the NCA ethyl acetate solution to effect a slow crystallization of NCA. The crystallization was repeated to obtain a sample with constant melting point and having an amount of hydrolysable chlorine lower than 0.05% by weight.

### EXAMPLE 2

Fully Protected Copolymer-1 Preparation

0.870 g of Ala-NCA, 0.596 g of Glu(OBzl)-NCA, 1.620 g of Lys(Z)-NCA and 0.450 g of Tyr(Bzl)-NCA were dissolved in 40 ml of dioxane to which 17 ml of diethyl amine in dioxane (5×10⁻⁴ g/ml) was added. The reaction mixture was stirred at room temperature for 48 hours. The reaction mixture was poured into 800 ml of water with good agitation. The white precipitate was filtered and washed subsequently with water and acetone. After drying in vacuum, 2.56 g (91.3% yields) of fully protected copolymer-1 was obtained. EXAMPLE 3 Copolymer-1 HBr Preparation

1.5 g of protected copolymer-1 was dissolved in 15 ml of 40% HBr/HOAc and stirred at 30° C. for 16 hours. The resulting reaction mixture was distilled under vacuum to remove HBr and acetic acid. The residue was extract five times with dichloromethane (10 ml each time) and then was washed three times with ether (10 ml each time) to give after vacuum drying 1.4 g of crude copolymer-1 HBr salt as a pale yellow powder.

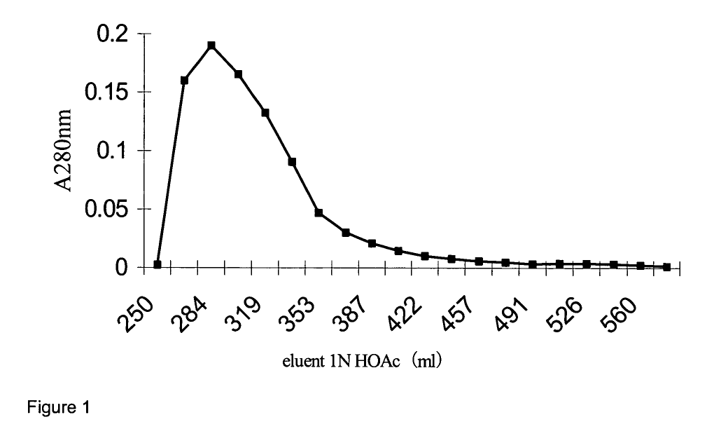

200 mg of crude copolymer-1 HBr was dissolved in 4 ml 1 N acetic acid, the resulting solution was loaded on a Sephadex G25 ((φ4.2×48 cm) column which was equilibrated with 1 N acetic acid. The elution between 243˜429ml (see FIG. 1) was collected and lyophilized to give 149 mg of copolymer-1 HBr.

### EXAMPLE 4

Copolymer-1 HOAc Solution Preparation

150 mg copolymer-1 HBr was dissolved in 3 ml water and cooled at an ice bath. To this solution, 0.15 ml of 10% Na₂CO₃ solution was added (pH8 ˜9), the pH of the solution was then adjusted to pH3 ˜4 by addition of 0.2 ml of acetic acid to give a copolymer-1 HOAc solution.

### EXAMPLE 5

Purification of Copolymer-1 Acetate

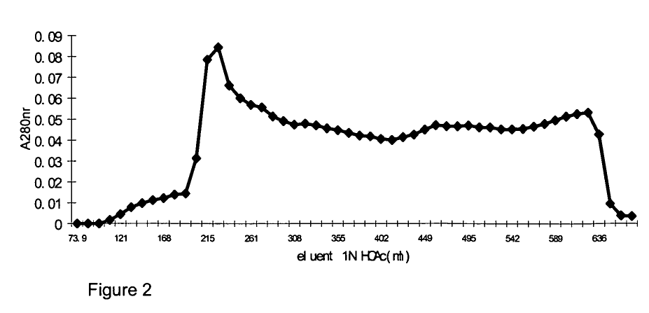

3 ml of copolymer HOAc solution (50 mg/ml in 1 N HOAc) was loaded on a Sephadex G50 (φ2.3×159 cm) column which was equilibrated with 1N acetic acid. The elution between 290˜490 ml (see FIG. 2) was collected and lyophilized to give 61.5 mg of copolymer-1 acetate with desired molecular weight distribution (copolymer-1 No. 200503 A) with a yield of 41%.

### EXAMPLE 6

Product Analysis: Copolymer-1 No. 200503 A

6.1 Amino acid composition analysis 0.1 mg copolymer-1 (No. 200503 A) was hydrolyzed in 2 ml of 6N HCI containing phenol at 110° C. for 18 hours. The resulting solution was analyzed by HITACHI 835 Amino Acid Analyzer. The amino acid molar ratio was shown in Table 1. The commercial copolymer-1 named Copaxone was used as a control.

TABLE 1
Amino acid composition of copolymer-1

| Amino acids | copaxone | No. 200503 A |
|---|---|---|
| Ala | 0.427 | 0.419 |
| Glu | 0.150 | 0.143 |
| Lys | 0.327 | 0.327 |
| Tyr | 0.100 | 0.103 |

6.2 Superdex 75 10/30 GPC Analysis The molecular weight distribution of copolymer-1 (No. 200503 A) was analyzed by Superdex 75 HR 10/30 and calculated using proteins as Mw markers. The mobile phase was 0.05M PBS containing 0.15 M NaCl, pH 7.0, detected at 230 nm. The data were shown in table 2. The commercial copolymer-1 named copaxone was used as a control.

TABLE 2
GPC analysis of copolymer-1 (No. 200503A) by Superdex 75

| | | | | % of MW | |
|---|---|---|---|---|---|
| | Mn | | Mp | 2,000˜20,000 | % of MW |
| sample | Da | Mw Da | Da | Da | >40,000 Da |
| copaxone | 5851 | 14566 | 16046 | 68 | 2.05 |
| No. 200503A | 8291 | 14941 | 15965 | 73.7 | 0.96 |

Thus, while there have shown and described and pointed out fundamental novel features of the invention as applied to a preferred embodiment thereof, it will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method for preparing copolymer-1 comprising reacting N-carboxy anhydrides of alanine, α-N-R₁-lysine, O-R₂-tyrosine and γ-R₃-glutamate with an initiator in a solvent medium to produce a protected copolymer-1, and deprotecting the protected copolymer-1 to produce copolymer-1, wherein the protecting group R₁, R₂ R₃ are organic groups which can be removed by base cleavage, acidolysis, thiolysis, hydrogenation or enzyme-catalyzed hydrolysis.

2. The method of claim 1 where in R₁, R₂, R₃ are alkyl groups of more than three carbon atoms and/or aromatic groups.

3. The method of claim 1, wherein the protecting group R₁ is selected from the group consisting of benzyloxycarbonyl group, 4-methoxybenzyloxycarbonyl group, α,α-dimethyl 3,5-dimethoxybenzyloxy group, 2-(4-biphenylyl) isopropoxycarbonyl group, t-butyloxycarbonyl group, 2,2,2-trichloroethoxycarbonyl group, t-amyloxycarbonyl group, adamantyloxycarbonyl group, allyloxycarbonyl group, o-nitrophenylsulfenyl group, trityl group, 9-fluorenylmethyloxycarbonyl group, phenylacetyl group, and pyroglutamyl group.

4. The method of claim 1, wherein the protecting group R₂ is selected from the group consisting of benzyl group, 2,6-dichlorobenzyl group, 2-bromobenzyloxycarbonyl group, t-butyl group, and 2,4-dinitrophenyl group.

5. The method of claim 1, wherein the protecting group R₃ is selected from the group consisting of cyclohexyl ester, benzyl ester, t-butyl ester, allyl ester, adamantyl group, 9-fluorenylmethyl group.

6. The method of claim 1, wherein said copolymer-1 is a mixture of polypeptides composed of alanine, glutamic acid, lysine, and tyrosine in a molar ratio of L-Ala:L-Glu:L-Lys:L-Tyr approximately 0.427:0.150:0.327:0.100, and the deviation may vary by about ±10%.

7. The method of claim 1, wherein the initiator is sodium methoxide or sodium t-butoxide.

8. The method of claim 1, wherein the initiator is an amine initiator.

9. The method of claim 8, wherein the amine initiator is selected from the group consisting of diethylamine, hexylamine, and phenethylamine.

10. The method of claim 1, wherein the initiator is a transition metal initiator.

11. The method of claim 10, wherein the transition metal initiator is bbyNi(COD) or (Pme3)4Co.

12. The method of claim 1, wherein the polymerization is carried out in an organic solvent selected from the group consisting of an ether, dioxane, tetrahydrofuran, dichloromethane, dimethylformamide, N-methylpyrrolidone, sulfolane, nitrobenzene, tetramethylurea and dimethylsulfone.

13. The method of claim 1, wherein the protected copolymer-1 is prepared from the N-carboxyanhydrides of O-benzyl-tyrosine, alanine, γ-benzyl-glutamate and ε-N-benzyloxycarbonyl-lysine.

14. The method of claim 1, wherein the protected copolymer-1 is prepared from the N-carboxyanhydrides of O-t-butyl-tyrosine, alanine, γ-t-butyl-glutamate and ε-N-t-butyloxycarbonyl-lysine.

15. The method of claim 13, protected copolymer-1 is prepared from the mixture of O-benzyl-tyrosine, alanine, γ-benzyl-glutamate and ε-N-benzyloxycarbonyl-lysine using triphosgene, phosgene or diphosgene and an initiator.

16. The method according to claim 14, wherein the protected copolymer-1 is prepared from the mixture of N-t-butyloxycarbonyl protected O-t-butyl-tyrosine, alanine, γ-t-butyl-glutamate and ε-N-t-butyloxycarbonyl-lysine using triethylamine/triphosgene, phosgene or diphosgene and an initiator.

17. The method of claim 1, wherein the deprotection of the protected copolymer-1 is effected by reaction with hydrogen bromide in glacial acetic acid.

18. The method of claim 1, wherein the deprotection of the protected copolymer-1 is effected by reaction with trifluoroacetic acid or hydrogen chloride in a solvent medium of acetic acid, dioxane or ethyl acetate.

19. The method of claim 1, wherein the solvent medium is an ether and the initiator is diethylamine.

20. The method of claim 1, wherein the copolymer-1 is purified through Sephadex G25 or Sephadex G50.
